(19) United States
(12) Patent Application Publication
(10) Pub. No.: US 2023/0248279 A1
(43) Pub. Date: Aug. 10, 2023

(54) BLOOD PRICKING HEAD FOR AN AUTOMATIC OR SEMI-AUTOMATIC BLOOD COLLECTION MACHINE

(71) Applicant: BEE HEALTHCARE, NANTES CEDEX 3 (FR)

(72) Inventors: Aliaume BRETEAU, SAINT-HERBLAIN (FR); Jean- Baptiste DE CHAISEMARTIN, SAINT-HERBLAIN (FR); Sylvain GEFFROY, SAINT-HERBLAIN (FR); Alexandre RIVIERE, SAINT-HERBLAIN (FR)

(73) Assignee: BHEALTHCARE, SAINT-HERBLAIN (FR)

(21) Appl. No.: 18/012,974

(22) PCT Filed: Jun. 23, 2021
(86) PCT No.: PCT/EP2021/067200

§ 371 Date: Dec. 26, 2022

(30) Foreign Application Priority Data
Jun. 26, 2020 (FR) FR2006743

(51) Int. Cl. A61B 5/15; A61B 34/30

(52) U.S. Cl. CPC A61B 5/15016; A61B 5/150748; A61B 5/150732; A61B 34/30

(57) ABSTRACT

The invention relates to a blood pricking head (8) for a mechatronic assembly such as a robot-controlled arm of an automatic or semi-automatic blood collection machine configured to enable movement of the mechatronic assembly below the limb of a patient, the pricking head comprising: a frame (10) provided with removable attachment means (11) that can be moved from the frame to the mechatronic assembly; a holder (12) for collection needles, means (16) for linearly moving the needle holder; a motorised device (25) for fluidically linking the needle mounted on the needle holder with a collection tube; and an electromechanical device (21, 22) configured to enable, on command, withdrawal of the pricking needle from the patient's arm.

### TECHNICAL FIELD OF THE INVENTION

The invention relates to a blood puncture head intended to equip an automatic or semi-automatic blood sampling machine. The invention also relates to an automatic or semi-automatic blood sampling machine which is provided with a blood puncture head according to the invention.

### TECHNICAL BACKGROUND

Health organisations carry out a very large number of blood tests each day. These operations are time-consuming, repetitive and potentially dangerous for the care staff and the patient taking into account the risks of injury which they involve and which are linked, for example, to shaking of the care staff, fatigue, inexperience, an incorrect movement or an incorrect reflex of the patient when the needle is introduced into their vein.

Furthermore, some patients have veins which are not very suitable for a good blood sample and which may involve several attempts to puncture by the care staff before being able to reach a vein which enables blood to be taken. This repetition of attempts may be painful for the patient and can cause injuries, which only complicates the sampling operation for these patients. US 2012/190981 discloses an autonomous intravenous insertion system comprising a robotic arm, one or more sensors which are fixed in a pivoting manner to the robotic arm in order to collect information relating to the potential insertion sites in an arm of a subject, a medical device which is fixed in a pivoting manner to the robotic arm, and a controller which communicates with the sensors and the robotic arm, in which the controller receives the information of the sensors relating to the potential insertion sites, and the controller selects a target insertion site and directs the robotic arm in order to insert the medical device into the target insertion site.

Furthermore, the worldwide Coronavirus health crisis requires the development of a system which limits contacts between patients and care staff and/or which can enable mass screenings of the population.

The Applicant has thus proposed in document WO2015158978 a machine for automatic puncture in a vein of a patient comprising means for infra-red imaging of the arm of the patient, means for detecting a vein in the image taken, a device for holding the detected vein, a needle and means for inserting the needle into the detected vein.

Such a device thus enables blood sampling operations to be automated.

However, some operations which are required for the blood sampling, such as the application of a dressing, control of collection tubes, checking the sample, etcetera, always require a human presence and/or additional equipment.

The Applicant has thus attempted to improve the proposed device and in particular to provide an autonomous puncture head which is intended to be provided on a mechatronic assembly such as a robotic arm of an automatic or semi-automatic blood sampling machine.

In the entire text, an automatic or semi-automatic machine is intended to be understood to be a machine which can operate without human intervention, with the exception of any loading operations of the machine, start-up of the machine and/or checking of the machine.

### OBJECTIVES OF THE INVENTION

The invention is intended to provide a blood puncture head which is intended to be provided on a mechatronic assembly, such as a robotic arm of an automatic or semi-automatic puncture machine.

The invention is intended in particular to provide an autonomous blood puncture head which can be provided on any type of mechatronic assembly, such as a robotic arm, of any type of automatic or semi-automatic blood sampling machine.

The invention is also intended to provide, in at least one embodiment of the invention, a blood puncture head which enables a sampling needle to be fitted and which enables the fluidic connection between the sampling needle and a tube for receiving the blood sample to be ensured automatically.

The invention is also intended to provide, in at least one embodiment of the invention, a blood puncture head which can be fitted to collection tubes which are intended to collect the blood samples.

The invention is also intended to provide, in at least one embodiment of the invention, a blood puncture head which enables the presence of blood in the collection tubes to be verified.

The invention is also intended to provide, in at least one embodiment of the invention, a blood puncture head which enables the filling of the collection tubes to be verified automatically.

The invention is also intended to provide, in at least one embodiment of the invention, a puncture head which enables the collection tubes to be changed automatically.

The invention is also intended to provide, in at least one embodiment of the invention, a blood puncture head which enables the bevel of the needle to be positioned correctly away from the skin of the limb of the patient to be punctured.

The invention is also intended to provide, in at least one embodiment of the invention, a blood puncture head which enables a dressing to be automatically applied to the skin of the patient following the sampling operation.

The invention is also intended to provide, in at least one embodiment of the invention, a blood puncture head which enables the blood puncture operation to be interrupted in the event of detected anomalies, and in particular enables the needle to be removed from the limb of the patient in the event of an electrical power failure.

The invention is also intended to provide, in at least one embodiment of the invention, a puncture head which enables automatic disinfection of the puncture zone.

The invention is finally intended to provide an automatic or semi-automatic blood sampling machine which is provided with a puncture head according to the invention.

### STATEMENT OF INVENTION

To this end, the invention relates to a blood puncture head which is intended to equip a mechatronic assembly, such as a robotic arm, of an automatic or semi-automatic blood sampling machine configured to allow movement of the head above a limb of a patient to be punctured.

The puncture head according to the invention comprises:
a frame equipped with means for removably fixing the frame to the mechatronic assembly,
a needle holder which is carried by the frame and which is adapted to receive a needle comprising a bevelled portion intended to pierce the skin of the patient's limb to be punctured and a rear portion intended to allow the flow of the collected blood to a collection tube.

The puncture head according to the invention is characterised in that it further comprises:
linear displacement means of the needle holder which are configured to allow, once the holder is equipped with a needle, the puncture head to be armed with a view to inserting, on command from the mechatronic assembly, this needle in the limb of the patient in order to be able to take a blood sample,
a device for fluidically connecting the rear portion of the needle mounted on the needle holder with a collection tube intended to collect the blood taken from the patient's limb,
an electromechanical device for disarming the head and emergency removal of the needle from the patient's limb configured to allow, on command, an exclusively mechanical removal of the puncture needle from the patient's limb.

A puncture head according to the invention thus has a needle holder, means for moving the needle holder, a device which is, for example, motorised, for placing the needle in fluidic connection with a collection tube (which may be fitted to the head as described above or arranged remotely from the head and connected to the needle using a connection means such as a flexible tube) and a device for disarming and emergency removal, which thus ensures an automated blood sample in a limb of a patient (which may be an upper limb of the patient, also referred to as the arm of a patient below or a lower limb of the patient). In other words, the various elements required for the blood sample of the patient move with the puncture head so that these elements are immediately accessible by the head, without requiring a movement of the mechatronic assembly towards an additional item of equipment or the intervention of an operator. A puncture head according to the invention can thus equip any robotic arm in order to form an automatic or semi-automatic puncture machine.

In the entire text, the terms “robotic arm” refer to a mechatronic assembly which is configured to enable the movement of the head carried by this mechatronic assembly above the limb of the patient to be punctured. In this manner, such a robotic arm may be formed by a shaft which is articulated, for example, along six axes, and which is controlled by a digital control (thus having the appearance of an arm as such) or any automated system which is provided with actuators (cables, electric motors, thrustors, supports, slides, etcetera) and which enables a load carried by the system to be moved on command (in this instance, the puncture head) towards the limb to be punctured.

In the entire text, the term “needle” is intended to refer to a slender rod which comprises a bevelled portion which is adapted to be able to pierce the skin of a patient and a rear portion which is adapted to be able to be placed in fluid communication with a blood collection tube or any consumable which is capable of collecting blood samples. Such a needle in the context of the invention may be a sampling needle per se but may also be any item of equipment which can be used for sampling, without nonetheless being initially intended for such sampling. For example, a needle in the context of the invention may be a cannula needle, a catheter, a micro infuser, etcetera, whose initial function is not blood sampling, but which can nonetheless be used for such a sampling. In the entire text, the terms “sampling needle” must be interpreted as a needle in the context of the invention.

A puncture head according to the invention also comprises a device for fluidically connecting the rear portion of the needle mounted on the needle holder with a collection tube intended to collect the blood taken from the patient's limb.

Such a device for fluidically connecting the rear portion of the needle and a collection tube may comprise a flexible tube which fluidically connects a collection tube which is arranged remotely from the puncture head and the rear portion of the sampling needle or directly an associated collection tube to means for securing/releasing the tube to/from the rear of the needle, as described below, or a connection member between a collection tube and a hose which is carried by the rear portion of the needle.

In the event that the collection tubes are not fitted to the puncture head, the fluidic connection device comprises, for example, a means for receiving a hose which is mounted on the rear portion of the sampling needle. This receiving means may, for example, be in the form of a cannula which is fixed to the rear of the needle and which is intended to fit into a hose which is connected to a collection tube which is arranged remotely from the sampling machine on which the puncture head according to the invention is mounted. This fitting is, for example, carried out by motorised means.

A puncture head according to the invention also comprises a needle holder which is configured to receive a needle and linear displacement means for the needle holder which thus enable the head to be armed on command in order to insert the needle into the limb of the patient by activating the mechatronic assembly.

The insertion of a needle into the limb of a patient is therefore carried out in two successive steps. In a first step, the puncture head is armed and the needle is placed in the puncture position by the movement means of the needle holder. In a second step, the mechanical assembly moves the head in a linear manner in the region of the determined puncture location and in the determined direction.

The operation for removing the needle, in a normal situation, is also carried out in two steps. In a first step, the head is moved in the opposite direction to the puncture direction until it leaves the limb of the patient. In a second step, once the needle is removed from the limb of the patient, the electromechanical removal device is activated in order to disarm the head.

In the event of an emergency, the removal of the needle is carried out in a single step which involves activating the electromechanical removal device in order to ensure a removal of the needle. This removal of the needle is ensured by exclusively mechanical means so that this emergency removal can be carried out including in the event of an absence of electrical power.

The electromechanical device for disarming the head and emergency removal therefore has a dual function. In other words, the same means are used both for the removal of the needle in a normal situation and for stopping the removal in the event of an emergency.

Furthermore and according to the invention, the fluidic connection device is motorised and comprises a device for loading and distributing blood collection tubes comprising at least one housing for receiving a collection tube and at least one securing/releasing actuator of this collection tube to/from the rear of a needle mounted on the needle holder, the securing/releasing actuator being configured to move the collection tube from the receiving housing to the rear portion of the needle, and vice versa.

In this manner, the collection tube is fitted to the head and can be moved from the receiving housing thereof at the rear of the needle by the securing actuator.

In other words, the tubes are fitted to the head and the fluidic connection is automatic and ensured by securing the collection tube to the rear of the tube.

Advantageously and according to the invention, the device for loading and distributing collection tubes comprises a barrel comprising a plurality of housings for receiving tubes distributed about an axis of rotation of the barrel, the axis of rotation extending parallel with the axis of the sampling needle, once the needle is mounted on the needle holder, and separated from this axis by a distance equal to the distance which separates the axis of rotation of the barrel from each of the housings for receiving the tubes so that each tube can be aligned with the needle, by rotation of the barrel, and secured at the rear of the needle, under the effect of the securing actuator formed by a linear actuator for moving the tube housed in the receiving housing aligned with the needle.

According to this advantageous variant, the barrel is mounted on the frame so that the housings for receiving the tubes may, by rotating the barrel, extend opposite the sampling needle.

A needle in the context of the invention is preferably formed by a bevelled vein needle which is intended to enter the vein of the limb of the patient to be punctured (which thus forms the bevelled portion of the sampling needle) and a stopper-piercing needle which extends in the axis of the bevelled needle and which is intended to extend into a collection tube in order to transport the collected blood there (which thus forms the rear portion of the sampling needle).

In this manner, a puncture head according to this variant of the invention enables several collection tubes to be filled without requiring the head to be moved in order to search for a new tube. This is because, after each filling of a tube, the filled tube can be separated from the stopper-piercing needle and repositioned in the barrel. This barrel can then pivot by a predetermined angle in order to position an empty tube opposite the needle, which is secured to the rear of the stopper-piercing needle in order to be able to collect a second sample of collected blood and so on for all the tubes of the barrel without having to retract the vein needle from the limb of the patient and without the intervention of an external operator.

Advantageously and according to this variant, the housings for receiving the collection tubes arranged at the periphery of the loading and dispensing barrel are loops for clip-fitting the tubes.

According to this variant, the collection tubes are mounted in the motorised loading and dispensing barrel for blood collection tubes by means of clip-fitting loops. This facilitates the loading of the tubes and also enables the tubes not to be obstructed (with the exception of the portion of the tube which is clamped in the clip-fitting loop). According to an advantageous variant, the receiving housings are distributed in a regular manner about the central rotation axis of the barrel.

Advantageously and according to the invention, the head further comprises a motorised dressing holder comprising a suction cup plate for holding a dressing, which plate is carried by a hollow shaft pivoting relative to the frame between a position for loading a dressing, in which the suction cup can come into contact with a dressing dispenser outside the head in order to be able to suck up a dressing by placing the hollow shaft under reduced pressure, and a position for applying the dressing, in which the dressing carried by the suction cup comes into pressed contact with the skin of the patient's limb in the region of the puncture zone, during withdrawal of the needle from the patient's limb in order to be able to affix the dressing to the patient's limb by placing the hollow shaft at atmospheric pressure, thereby releasing the dressing.

A puncture head according to this advantageous variant of the invention enables a dressing to be applied automatically in the region of the puncture zone whilst producing a compression location which enables any risk of internal or external bleeding to be prevented. This dressing applicator comprises a suction cup which is configured to be able to suck a dressing provided by a dressing distributor (external to the head and arranged in the region of the patient to be punctured) by placing a hollow tube which carries the suction plate under reduced pressure. To this end, the suction cup preferably comprises a plurality of holes which are arranged on the upper surface thereof in fluidic communication with the hollow tube so that placing the hollow tube under reduced pressure enables, as a result of the pressure difference, a dressing which is placed in contact with the upper surface of the suction cup to be drawn in, by moving the suction cup into the loading position. Preferably, the dressing distributor is loaded with a roll of dressings which is formed by a substrate on which the dressings which are spaced apart from each other by a predetermined distance are placed. Of course, other configurations are possible without calling into question the principle of this variant. The suction cup comes into contact with a dressing, sucks the dressing and the dressing applicator is ready to be used. The dressing distributor is preferably motorised so that, after sucking a dressing, it is rotated by a predetermined angle in order to present a dressing which is available for the next puncture. Preferably, the dressings are non-porous and have a rigidity with respect to the substrate in order to facilitate suction by the suction cup.

The hollow shaft of the dressing applicator is pivotably mounted relative to the frame between the position for loading a dressing and the position for applying the dressing. In this manner, the dressing applicator can, in the position for applying the dressing, apply a predetermined pressure to the limb of the patient by accentuating slightly the pivoting of the hollow shaft. The position for applying the dressing is, for example, determined by the extent of a resistant torque in the region of the electrical actuator which ensures the pivoting of the hollow shaft.

The upper surface of the suction cup may according to an advantageous variant of the invention have a recess allowing the passage of the needle during the application of the dressing. This variant thus enables the operations for applying the dressing and the removal of the needle to be carried out simultaneously.

Preferably the suction cup is pivotable with respect to the tube which enables it to adapt to the curvature of the patient's limb to be punctured, independently of the rotation and/or inclination of the puncture head.

Advantageously and according to the invention, the frame comprises a fixed portion which is fitted with means for removable fixing to the mechatronic assembly and a movable portion carrying at least the needle holder and the fluidic connection device, and in that the electromechanical device for emergency withdrawal comprises an electromagnet and a return means which extends between the fixed portion of the frame and the movable portion of the frame and which is configured to ensure the return of the movable portion of the frame towards the fixed portion of the frame when the electromagnet is no longer supplied with current.

An electromechanical device of a head according to this variant thus forms a safety device which enables immediate withdrawal of the needle from the arm of the patient in the event of an emergency.

During normal operation, the needle is withdrawn from the skin of the patient at the end of the puncture operation by activating the movement means of the needle holder. The electromechanical device is then activated in order to disarm the head.

In the event of an emergency, the withdrawal of the needle is ensured by a movement of the movable portion of the frame relative to the fixed portion of the frame which is brought about by the electromagnet, which brings about the complete disengagement of the needle by activating the return spring.

Advantageously and according to the invention, the head further comprises means for determining the filling level of the collection tube in fluidic connection with the sampling needle.

This advantageous variant enables the filling level of the tubes to be determined, which enables in particular a real time monitoring of the puncture. These means for determining the filling level may, for example, be formed by optical means for detecting the presence of blood in the filling tube which is in fluidic connection with the sampling needle. These means may also be formed by means for weighing the tube which enable the quantity of blood present in the tube to be determined from a knowledge of the empty weight of the tube. These means may also be formed by means for calculating the filling time which enable the quantity of blood present in the tube to be estimated from the puncture time and information representing the flow rate of the blood sampling operation. Of course, other means may be used to determine the filling level of the collection tube.

This variant also enables the filling of the tubes to be detected and therefore enables control of the change of a filled tube for an empty tube to be filled as soon as the filling level reaches a predetermined level.

In particular and advantageously, the means for determining the filling level of the collection tube are configured to be able to trigger an automatic change of the collection tube.

For example, if the head is equipped with a barrel for loading and distributing tubes, the detection of a specific filling level of the tube secured to the needle brings about the movement of the filled tube in the receiving housing thereof, the rotation of the barrel in order to align an empty tube at the rear of the needle, then the movement of this empty tube in order to ensure the securing of the tube to the rear of the needle. This enables the blood sampling to be continued with a new tube without any external intervention. The process may be repeated for all the tubes provided for the examination carried out.

In other words, according to this variant, the detection of a predetermined filling level in a collection tube controls the securing/releasing actuator of this collection tube at the rear of a needle which is mounted on the needle holder and controls the changing of the tube (rotation of the barrel if the tube loading device comprises a barrel).

Advantageously and according to the invention, the head further comprises means for detecting the entry of the needle mounted on the needle holder into a vein of the patient to be punctured.

This advantageous variant enables the movement of the needle in the limb of the patient to be punctured to be controlled and in particular enables the time when the needle is in the vein to be punctured to be determined.

Advantageously and according to the invention, the head further comprises sensors which are arranged in the region of the needle holder and which are configured to detect an abnormal presence of blood in this region.

These sensors are, for example, resistive, capacitive or inductive sensors which are configured to enable the presence of blood to be detected in the region of zones which should not, during nominal operation, be affected by blood. These zones are adjacent to the fluid blood collection circuit.

Advantageously and according to the invention, the needle-holder comprises means for fixing by screwing, clip-fitting or magnetisation of the sampling needle on the needle-holder.

In the case of screw fixing means, they are, for example, formed by a dual-thread nut, each thread being conformed to the pitch of a screw of a type of needle which is commercially available. In this manner, the head according to the invention can receive different types of needles which are distinguished from each other, in particular by the shape of the thread.

Preferably, the needle holder further comprises a system for disengaging the rotation of the needle so as to allow rotation of the needle about itself in order to be able to orientate the bevel of the needle away from the skin of the patient's limb to be punctured.

This preferential feature enables the bevel to be orientated correctly in order to facilitate the insertion of the needle into the skin of the limb of the patient.

Advantageously and according to the invention, the head further comprises means for automatically disinfecting the puncture zone.

These automatic disinfection means comprise, for example, a spray which is fitted to the head, a system for placing a cotton pad soaked with disinfectant products or any equivalent means.

The invention also relates to a machine for automatic or semi-automatic blood sampling of a patient's limb comprising a mechatronic assembly, a control unit of the mechatronic assembly, a system for capturing and processing images of the patient's limb configured to determine an optimal puncture zone, characterised in that it further comprises a puncture head according to the invention mounted on the mechatronic assembly.

A machine according to the invention enables blood sampling to be automated in a limb of a patient. This machine comprises, in addition to the puncture head according to the invention, a mechatronic assembly such as a robotic arm, a control unit which enables the position of the robotic arm to be controlled and a system for capturing images and processing these images which is configured to enable an optimum puncture location to be defined. This optimum puncture location is then supplied to the control unit of the robotic arm and to the control unit of the puncture head in order to enable the needle to be inserted into the arm of the patient in the region of the optimum puncture zone determined by the image processing system. This image processing system is, for example, the one described in the application WO2015158978 in the name of the Applicant.

The image processing system may also be fitted directly to the puncture head according to the invention which is, for example, carried by the frame, in order to be able to acquire the limb of the patient to be punctured and to determine the optimum puncture zone. This variant enables the head to have all the functionalities required for the puncture, the main function of the blood sampling machine being to control the movements of the mechatronic assembly which carries the puncture head.

The advantages and effects described in connection with the puncture head apply mutatis mutandis to the sampling machine according to the invention.

The invention also relates to a puncture head and an automatic or semi-automatic blood sampling machine which are characterised in combination by all or some of the features mentioned above or below.

### LIST OF FIGURES

Other objectives, features and advantages of the invention will be appreciated from a reading of the following description, given purely by way of non-limiting example and with reference to the appended Figures, in which:

FIG. 1 shows a schematic, perspective front view of a puncture head according to an embodiment of the invention,

FIG. 2 shows a schematic, perspective view according to another orientation of the puncture head according to the embodiment of FIG. 1,

FIG. 3 shows a schematic, perspective view according to another orientation of the puncture head of the embodiment of FIGS. 1 and 2,

FIG. 4 shows a schematic side view of a needle holder which is equipped with a puncture needle of a puncture head according to an embodiment of the invention,

FIG. 5 shows a schematic view of an automatic blood sampling machine according to an embodiment of the invention.

### DETAILED DESCRIPTION OF AN EMBODIMENT OF THE INVENTION

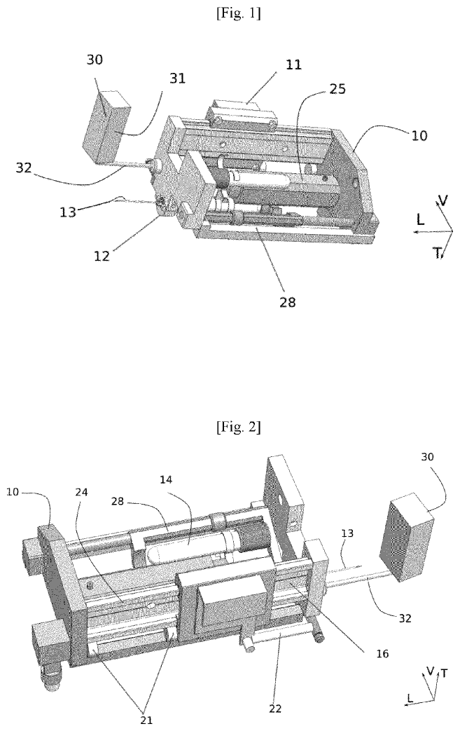

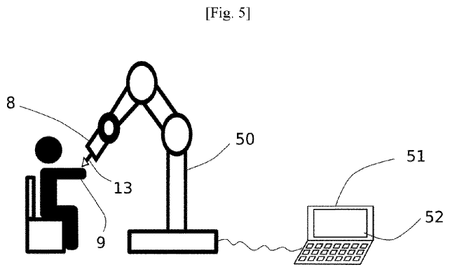

In the Figures, the scales and proportions are not strictly complied with for the purposes of illustration and clarity. In the entire detailed description which follows with reference to the Figures, unless indicated otherwise, each element of the puncture head according to the invention is described as it is when the head is mounted on a mechatronic assembly such as a robotic arm of an automatic sampling machine. The robotic arm is not, however, illustrated in FIGS. 1 to 4 for the purposes of clarity. The arm is illustrated schematically in FIG. 5.

Furthermore, elements which are identical, similar or comparable are given the same reference numerals in all the Figures.

Finally, the terms longitudinal, transverse, vertical and the variants thereof are used by way of non-limiting example with reference to the trihedron L, T V, as illustrated in particular in FIG. 1. The longitudinal direction (designated L in the Figures) corresponds to the main direction of the puncture head and coincides in particular with the direction along which the blood sampling needle extends when it is mounted on the needle holder. The vertical direction (designated V in the Figures) is the direction defined by gravitational force and the transverse direction (designated T in the Figures) is the direction perpendicular to the longitudinal direction and the vertical direction).

The blood puncture head according to the embodiment of the Figures comprises a frame 10 which is provided with means 11 for removably fixing the frame 10 to a robotic arm of an automatic blood sampling machine which is not illustrated in FIGS. 1 to 4 for the purposes of clarity. These fixing means 11 may be of any type and are dependent on the robotic arm on which the puncture head is mounted.

The puncture head also comprises a sampling needle holder 12 which is carried by the frame 10 and which is configured to receive a needle 13, such as a blood sampling needle. This needle holder 12 is illustrated in greater detail in FIG. 4, which also illustrates a collection tube 14 which is secured to the rear of the needle. The sampling needle 12 is formed by a bevelled portion 13a which is intended to enter the vein of the limb of the patient to be punctured and a stopper-piercing portion 13b which extends in the axis of the bevelled portion 13a and which passes through a stopper 15 in order to extend into the collection tube 14. This bevelled portion 13a and this stopper-piercing portion 13b may be formed from one and the same needle or two needles which are connected to each other.

In the remainder of the text, reference is made to a bevelled needle and a stopper-piercing needle, it being understood that they may be produced by one and the same needle as indicated above. The bevelled needle 13a and stopper-piercing needle 13b are in fluidic communication so that the blood removed by the bevelled needle 13a flows to the stopper-piercing needle 13b, then to the collection tube 14 which is secured to the rear of the stopper-piercing needle.

The sampling needle 13 is mounted on the needle holder 12 by means of screwing, clip-fitting or magnetisation.

According to one embodiment of the invention, the needle holder 12 also comprises means for disengaging the needle 13 which enable the needle 13 to be pivoted about the main axis thereof in order to enable the bevel of the needle to be orientated away from the skin of the patient to be punctured which facilitates the insertion thereof into the patient's limb to be punctured.

This disengagement is, for example, carried out by means of a thread of the needle holder which is pivotably mounted about the axis of the needle and which is held pressed against a retention plate by a spring. The force of this spring, the tension thereof and the nature of the materials used are configured to generate a controlled friction, which enables an adequate screwing torque to be obtained in order to ensure an optimum blocking in order to screw the needle, a rotational driving of the thread which ensures the correct positioning of the bevel of the needle and the absence of movement of the needle when the needle is inserted.

The needle holder 12 is fixedly joined to the frame 10 of the head using any possible mechanical means.

The puncture head also comprises linear displacement means 16 of the needle holder 12 which are configured, once the needle holder 12 is equipped with a sampling needle 13, to enable the head to be armed in order to insert this needle into the limb of a patient to be punctured.

These linear displacement means of the needle holder are preferably formed by the robotic arm which carries the puncture head when the needle is inserted into the limb of the patient and the robotic arm and an electromechanical disarming system (or triggering system) when the needle is removed.

This electromechanical triggering system is illustrated schematically in FIG. 2. It comprises in particular an electromagnet 21 and a return spring 22 which is configured to ensure the return of the needle holder along slides 24 when the electromagnet 21 is no longer supplied with current.

During normal operation, a magnetic suction cup which forms the electromagnet is supplied with current so that the return spring 22 is held in a stretched state. The triggering system is kept in this configuration for the entire duration of the puncture operation. In order to start the system, at the end of the puncture operation, in the event of emergency removal or in the event of a power failure, the magnetic suction cup releases the movable portion of the head which is then displaced by the force of the spring 22 along the slides as far as a damping stop.

According to one embodiment of the invention which is not illustrated in the Figures, the needle holder 12 may also comprise a needle presence sensor which is adapted to detect the presence of a needle on the needle holder. Such a sensor may, for example, be an optical sensor, a mechanical sensor, a magnetic sensor, or any equivalent means.

According to another embodiment which is not illustrated in the Figures, the frame comprises a fixed portion which is equipped with means for removably fixing to the robotic arm and a movable portion which carries the needle holder, the movable portion being configured to be able to be moved, on command from a control unit and associated actuators, relative to the fixed portion in order to ensure the insertion/removal of the needle carried by the needle holder in the limb of the patient, the movable portion thus forming the linear displacement means of the needle holder.

According to the embodiment of the Figures, the puncture head also comprises a motorised barrel 25 for loading and distributing blood collection tubes 14.

This barrel 25 comprises a plurality of housings 27 for receiving tubes 14 which are distributed about a rotation axis 26 of the barrel which forms a star-like barrel. Each receiving housing 27 is formed by a resilient clip-fitting handle. According to other embodiments, each receiving housing may be formed by a dual clip-fitting which enables the retention to be improved and the alignment of the tubes to be ensured.

The rotation axis 26 of the barrel extends parallel with the axis of the sampling needle 13, after the needle has been mounted on the needle holder 12, but is offset relative to the axis of the needle. This rotation axis 26 is rotationally driven by an electric motor which cannot be seen in the Figures. This electric motor is preferably accommodated in the rotation axis 26. Of course, other electromechanical means may be used in order to ensure this rotation of the rotation axis 26 of the barrel 25.

The rotation of the barrel 25 about the rotation axis 26 enables each collection tube 14 which is accommodated in a receiving housing 27 to be positioned in turn in the axis of the needle 13. Once the collection tube 14 is in the axis of the needle 13, the tube is moved, under the action of a bidirectional linear actuator 28, towards the rear of the needle 13 in order to be secured at that location. After the tube has been secured to the rear of the needle 13, the blood sampling can begin. The linear actuator 28 being bidirectional, it can also ensure the removal of the collection tube, after it has been filled, in order to be replaced with another tube of the barrel 25.

The puncture head 8 according to the embodiment of the Figures also comprises a motorised dressing applicator 30 which comprises a suction cup plate 31 for holding a dressing carried by a hollow shaft 32 which pivots relative to the frame 10. Such a bidirectional actuator is, for example, a rack and pinion actuator.

The pivoting of the hollow shaft 32 enables the suction cup plate to be displaced between a position for loading a dressing in which the suction cup may come into contact with a dressing distributor outside the head in order to be able to suck a dressing by placing the hollow shaft 32 under reduced pressure, and a position for applying the dressing, in which the dressing carried by the suction cup comes into contact with the skin of the limb of the patient in the region of the puncture zone, when the needle 13 is withdrawn from the limb of the patient in order to be able to apply the dressing to the limb of the patient by placing the hollow shaft 32 at atmospheric pressure, thus releasing the dressing.

The pivoting of the hollow shaft 32 is ensured, for example, by an electric motor which rotationally drives a gear washer which meshes with a gear washer which is fixedly joined to the hollow shaft 32. Of course, other electromechanical means may be used in order to ensure the pivoting of the hollow shaft from the position for drawing in the dressing to the position for applying the dressing to the limb of the patient.

According to one embodiment of the invention which is not illustrated in the Figures, the dressing applicator 30 may also comprise a sensor for detecting a dressing. Such a sensor may, for example, be an optical sensor, a mechanical sensor, a reduced pressure sensor or any equivalent means.

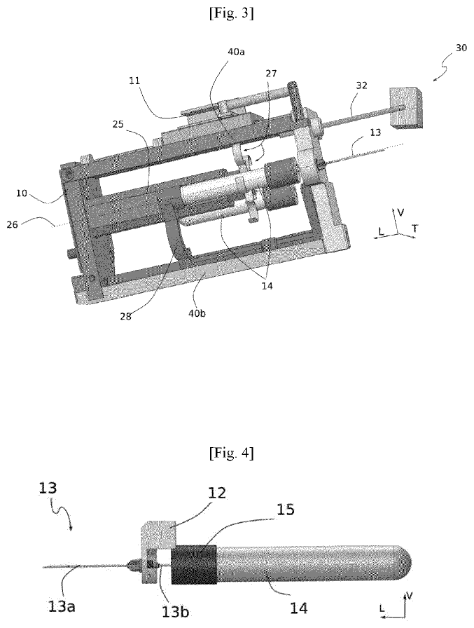

The puncture head preferably also comprises, as illustrated in FIG. 3, means 40a, 40b for controlling the filling, the presence and the positioning of the tubes. These filling control means 40a, 40b comprise, according to the embodiment of the Figures, optical sensors which are configured to detect the presence of blood in the collection tubes 14 and the filling level of the tubes. It is thus possible to monitor in real time the progress of the blood sampling. These sensors also enable the presence and the nominal positioning of the tube to be detected before puncture.

All types of optical sensors may be used to carry out this detection of blood in the tube and the filling level of the tube. According to the embodiment of the Figures, a filling means 40a is arranged at the output side of the tubes and a filling means 40b is arranged at the receiving side of the tubes.

The puncture head according to the embodiment of the Figures also comprises resistive sensors which are arranged in the region of the needle holder 12 and the barrel 25 for loading and distributing the tubes and which are configured to detect an abnormal presence of blood in this region.

The puncture head also comprises a control unit which is configured to receive the data and/or measurements of the various sensors and actuators of the head which, together with the mechatronic assembly, enable the operation and the movement of the puncture head to be controlled.

The invention also relates to an automatic blood sampling machine which is illustrated very schematically in FIG. 5. This machine comprises, according to the embodiment illustrated, a robotic arm 50, a control unit 51 of the robotic arm, a system 52 for capturing and processing images of the limb of the patient which is configured to determine an optimum puncture zone, and a puncture head 8 according to the embodiment of FIGS. 1 to 4. The system 52 for capturing and processing images comprises, for example, a camera which is not illustrated in the Figures and which is capable of carrying out the acquisition of the limb of the patient 9 to be punctured. The images are then transmitted to an image processing module which is configured to detect an optimal puncture vein. The method for identification of the optimal vein is, for example, the method described in the application WO 2015158978 in the name of the same Applicant. After the optimum vein has been detected, the control unit 52 transmits to the actuators of the robotic arm 50 and to the actuators of the puncture head 8 the movement information items of the needle holder in order to enable the insertion of the needle 13 into the limb of the patient 8 to be sampled.

Prior to the blood sampling operation, the puncture head according to the invention is loaded with the collection tubes necessary for the specific analysis of the patient. The tubes are inserted into the rotating barrel, which preferably comprises a number of tube receiving housings which is greater than or equal to the number of tubes required for the examination carried out on the patient.

The head is then loaded with a dressing. As explained above, this loading of the dressing may be carried out by the displacement of the hollow arm 32 which carries the suction cup plate 31 to the position for loading a dressing, in which the suction cup may come into contact with the dressing distributor.

According to another variant, the dressing distributor may be replaced by an operator who places the dressing directly on the suction cup plate 31. In the same manner, the operations for loading tubes, assembling needles or disinfection may, according to a variant, be carried out by an operator.

As soon as the system for capturing and processing images has defined the optimum puncture location, the puncture head is placed in a sampling position by the robotic arm. The head advances towards the limb of the patient to be punctured and the needle penetrates the skin in accordance with a predetermined angle, either via the target vein. The first collection tube conforming to the regulations is then inserted in the stopper-piercing needle in accordance with the mechanism described above.

The filling of the tube is carried out and when a predetermined filling level is reached (detected by the means for controlling the filling of the tubes), the filled tube is replaced with an empty tube of the barrel. This cycle of operations is repeated until the intended samples have been completed for the patient in question.

After all the samples have been carried out, the needle is withdrawn from the limb of the patient and the dressing present on the suction cup is applied to the limb of the patient in the region of the puncture location.

1. A blood puncture head intended to equip a mechatronic assembly such as a robotic arm, of an automatic or semi-automatic blood sampling machine configured to allow movement of said head above a limb of a patient to be punctured, said blood puncture head comprising:
a frame equipped with means for removably fixing the frame to said mechatronic assembly, and
a needle holder carried by said frame and adapted to receive a needle comprising a beveled portion intended to pierce the skin of the patient's limb to be punctured and a rear portion intended to allow the flow of collected blood to a collection tube, wherein said blood puncture head further comprises:
linear displacement means of said needle holder configured to allow, once equipped with a needle, to arm the puncture head with a view to inserting, on command from said mechatronic assembly, this needle in said limb of said patient in order to be able to take a blood sample,
a device for fluidically connecting said rear portion of said needle mounted on said needle holder with a collection tube adapted to collect the blood taken from said patient's limb, and
an electromechanical device for disarming the head and emergency removal of said needle from said patient's limb configured to allow, on command, an exclusively mechanical removal of said needle from said patient's limb.

2. The puncture head according to claim 1, wherein said fluidically connecting device is motorized and further comprises a device for loading and distributing blood collection tubes comprising at least one housing for receiving a collection tube and at least one securing actuator of this collection tube to the rear of a needle mounted on said needle holder, said securing actuator (being configured to move said collection tube from the receiving housing to the rear portion of said needle and vice versa.

3. The puncture head according to claim 2, wherein said device for loading and distributing sampling tubes comprises a barrel comprising a plurality of housings for receiving tubes distributed around an axis of rotation of said barrel, said axis of rotation extending parallel to the axis of said needle, once the needle is mounted on said needle holder, and separated from this axis by a distance equal to the distance which separates said axis of rotation of said barrel from each of said housings for receiving said tubes, so that each tube can be aligned with said needle, by rotation of said barrel, and secured at the rear of said needle, under the effect of said securing actuator formed by a linear actuator for moving the tube housed in the receiving housing aligned with said needle.

4. The puncture head according to claim 3, wherein said housings for receiving the sampling tubes formed at the periphery of said loading and dispensing barrel comprise loops for clipping said tubes.

5. The puncture head according to claim 1, wherein it further comprises a motorized dressing holder comprising a suction cup plate for holding a dressing carried by a hollow shaft pivoting relative to said frame between a position for loading a dressing in which said suction cup can come into contact with a dressing dispenser outside said head in order to be able to suck up a dressing by placing said hollow shaft under vacuum, in a position for placing the dressing, in which said dressing carried by said suction cup comes into pressed contact with the skin of said patient's limb at the level of the puncture zone, during withdrawal of said needle from said patient's limb in order to be able to affix said dressing on said patient's limb by venting said hollow shaft, thereby releasing said dressing.

6. The puncture head according to claim 5, wherein said suction cup plate of the dressing holder comprises a recess allowing the passage of the needle during the fitting of the dressing.

7. The puncture head according to claim 5, wherein said suction plate is pivotable with respect to the hollow shaft so as to be able to adapt to the curvature of the patient's limb at puncture, independent of the rotation and/or inclination of the puncture head.

8. The puncture head according to claim 1, wherein said frame comprises a fixed part fitted with means for removable fixing to said mechatronic assembly and a mobile part carrying at least said needle holder and said fluidic connection, and in that said electromechanical device for disarming and emergency withdrawal comprises an electromagnet and a return means extending between the fixed part of said frame and said movable part of said frame, and configured to ensure the return of the movable part of said frame to the fixed part of said frame when said electromagnet is no longer supplied with current.

9. The puncture head according to claim 1, wherein it further comprises means for determining the filling level of the collection tube in fluidic connection with said sampling needle.

10. The puncture head according to claim 9, wherein said means for determining the filling level of the collection tube are configured to be able to trigger an automatic change of the sampling tube.

11. The puncture head according to claim 1, wherein it further comprises means for detecting the entry of said needle mounted on said needle holder into a vein of the patient to be punctured.

12. The puncture head according to claim 1, wherein said needle-holder comprises means for fixing by screwing, clipping or magnetization of said puncture needle on said needle-holder.

13. The puncture head according to claim 12, wherein said needle holder further comprises a system for disengaging the rotation of said needle so as to allow rotation of the needle on itself in order to be able to orient the bevel said needle away from the skin of the patient's limb to be punctured.

14. The puncture head according to claim 1, wherein it also comprises means for the automatic disinfection of said puncture zone.

15. A machine for automatic or semi-automatic puncture of a patient's limb comprising a mechatronic assembly, a control unit of said mechatronic assembly, a system for capturing and processing images of the patient's limb configured to determine an optimal puncture zone, wherein the machine further comprises a puncture head mounted on said mechatronic assembly, the puncture head comprising:
a frame equipped with means for removably fixing the frame to said mechatronic assembly, and
a needle holder carried by said frame and adapted to receive a needle comprising a beveled portion intended to pierce the skin of the patient's limb to be punctured and a rear portion intended to allow the flow of collected blood to a collection tube, wherein said blood puncture head further comprises:
linear displacement means of said needle holder configured to allow, once equipped with a needle, to arm the puncture head with a view to inserting, on command from said mechatronic assembly, this needle in said limb of said patient in order to be able to take a blood sample,
a device for fluidically connecting said rear portion of said needle mounted on said needle holder with a collection tube adapted to collect the blood taken from said patient's limb, and
an electromechanical device for disarming the head and emergency removal of said needle from said patient's limb configured to allow, on command, an exclusively mechanical removal of said needle from said patient's limb.
